(19) United States
(12) Patent Application Publication
(10) Pub. No.: US 2024/0238128 A1
(43) Pub. Date: Jul. 18, 2024

(54) INCISION DRESSING

(71) Applicant: DeRoyal Industries, Inc., Powell, TN (US)

(72) Inventors: Gavin L. Warrington, Powell, TN (US); Thomas Paul Lawhorn, Sevierville, TN (US); Ethan Edward Valentine, Knoxville, TN (US); Dhanvin Sunil Desai, Knoxville, TN (US)

(73) Assignee: DeRoyal Industries, Inc., Powell, TN (US)

(21) Appl. No.: 18/154,192

(22) Filed: Jan. 13, 2023

(51) Int. Cl. A61F 13/02; A61L 15/22; A61L 15/28; A61L 15/42; A61L 15/58

(52) U.S. Cl. CPC A61F 13/0206; A61L 15/225; A61L 15/28; A61L 15/425; A61L 15/58

(57) ABSTRACT

The disclosure provides incision dressings having a liquid permeable center section; a pair of lateral absorbent sections abutting the center section on each side of the center section; and a wound contact material adhered to lowermost surfaces of the center section and the lateral absorbent sections.

### FIELD

The present disclosure relates to medical dressings. More particularly, the disclosure relates to improved dressings having improved structure and aesthetics, and configured for use at incision sites and other wound sites typically having a higher incident of exudate or fluid discharge.

### BACKGROUND

Improvement is desired in the provision of medical dressings for incision sites. What is desired is a dressing that is of simple construction and better configured to handle exudate and wound discharge commonly associated with incision sites.

The present disclosure advantageously provides dressings constructed to provide advantageous treatment of wound exudate. The dressings also have improved aesthetics as compared to conventional dressings with a unique appearance.

### SUMMARY

The disclosure advantageously provides incision dressings.

In one aspect, an incision dressing according to the disclosure includes a liquid permeable center section; a pair of lateral absorbent sections abutting the center section on each side of the center section; and a wound contact material adhered to lowermost surfaces of the center section and the lateral absorbent sections.

In another aspect, an incision dressing according to the disclosure includes a liquid permeable center section; an absorbent section abutting and surrounding the center section; and a wound contact material adhered to lowermost surfaces of the center section and the absorbent section.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 is an exploded perspective view showing an incision dressing according to the disclosure.

FIGS. 2A and 2B are upper and lower perspective views of the incision dressing without the adhesive covering and the release liners.

FIGS. 3, 4 and 5 show the incision dressing with an adhesive covering and release liners applied, with FIG. 3 being a top view, FIG. 4 being a side view, and FIG. 5 being an upper perspective view.

FIGS. 6A and 6B depict application of the incision dressing with the adhesive covering to an incision.

FIGS. 7A and 7B are upper and lower perspective views of the incision dressing provided in a square shape and without the adhesive covering and the release liners.

FIGS. 8A and 8B are upper and lower perspective views of the incision dressing provided in a circular shape and without the adhesive covering and the release liners.

FIGS. 8A and 8B are upper and lower perspective views of the incision dressing provided in a circular shape and without the adhesive covering and the release liners.

FIGS. 9A and 9B are upper and lower perspective views of the incision dressing provided in a circular shape and without the adhesive covering and the release liners.

FIGS. 10A and 10B are upper and lower perspective views of the incision dressing provided in an oval shape and without the adhesive covering and the release liners.

FIGS. 11A and 11B are upper and lower perspective views of the incision dressing provided in a C-shape and without the adhesive covering and the release liners.

FIGS. 12A and 12B are upper and lower perspective views of the incision dressing provided in a T-shape and without the adhesive covering and the release liners.

FIGS. 13A and 13B are upper and lower perspective views of the incision dressing provided in a triangular shape and without the adhesive covering and the release liners.

FIGS. 14A and 14B are upper and lower perspective views of the incision dressing provided in a U-shape and without the adhesive covering and the release liners.

FIGS. 15A and 15B are upper and lower perspective views of the incision dressing provided in a Y-shape and without the adhesive covering and the release liners.

### DETAILED DESCRIPTION

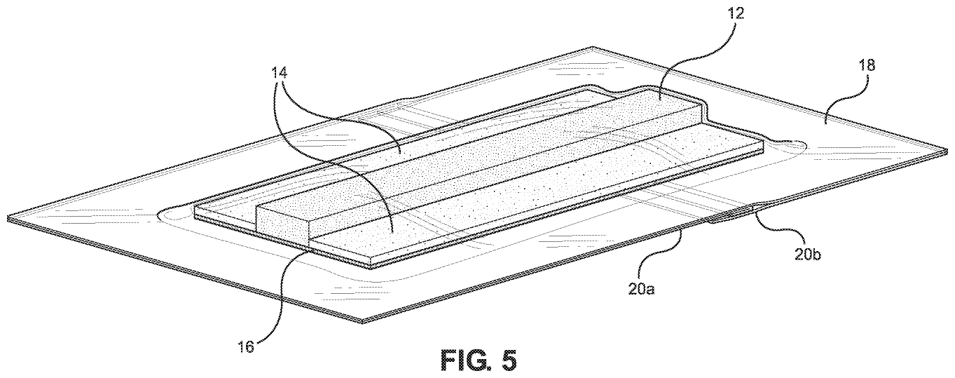

With reference to the drawings, the disclosure provides an incision dressing 10 of improved construction. The incision dressing 10 is configured to have improved fluid transport properties to transport wound fluids away more effectively from a wound. As described and shown herein the wound is an incision I such as a surgical incision (FIG. 6A). It will be understood that the dressing 10 may be applied to wounds other than incisions. The appearance of the dressing 10 is also configured to be aesthetically pleasing and includes various ornamental aspects and features.

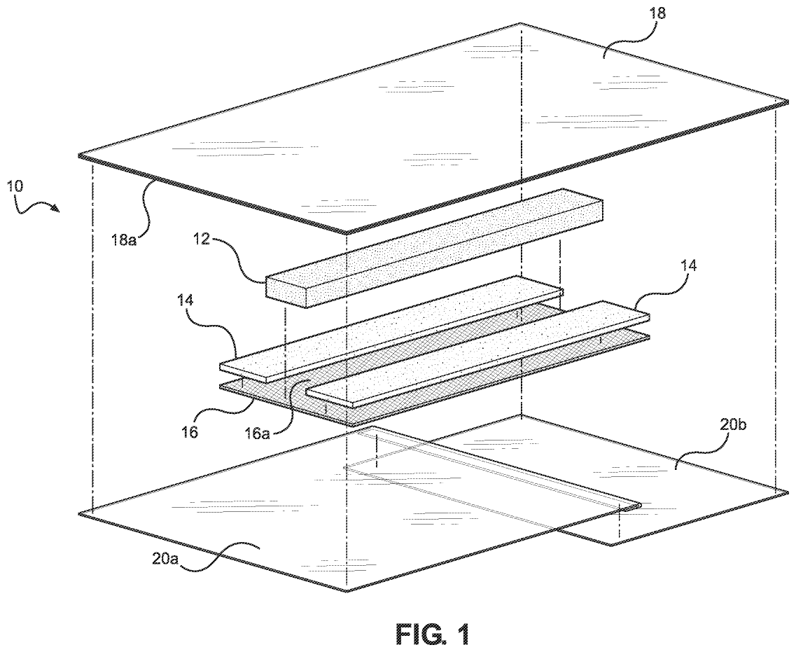

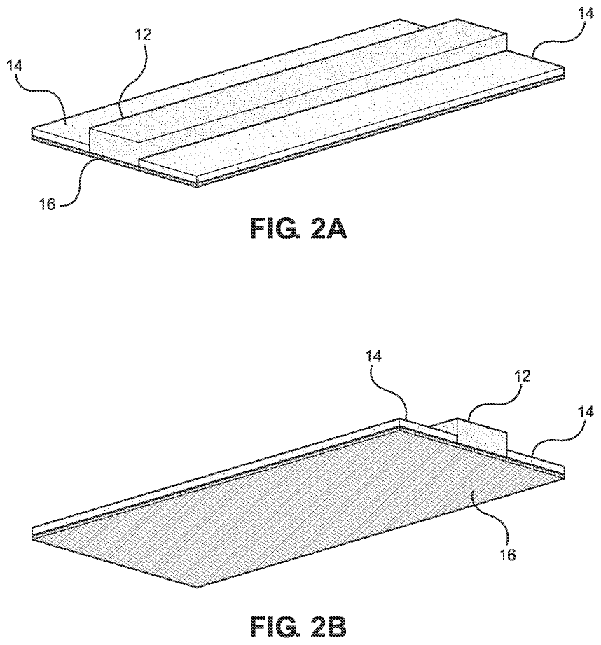

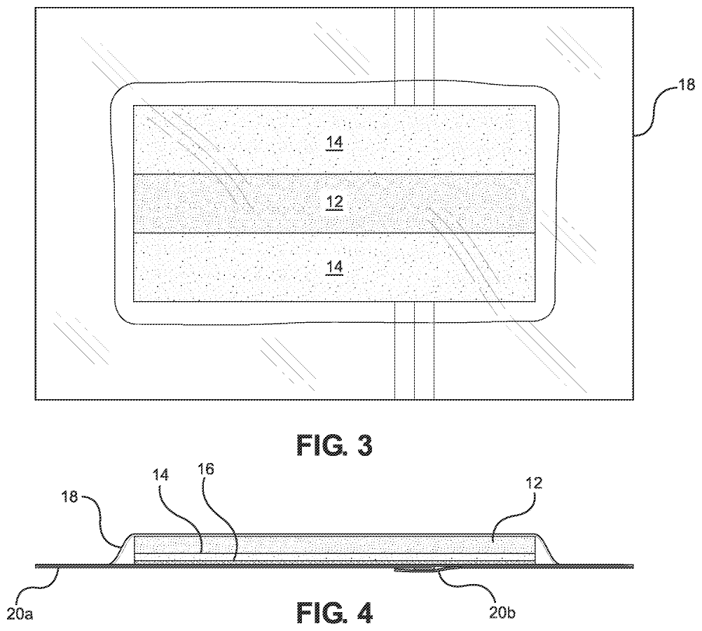

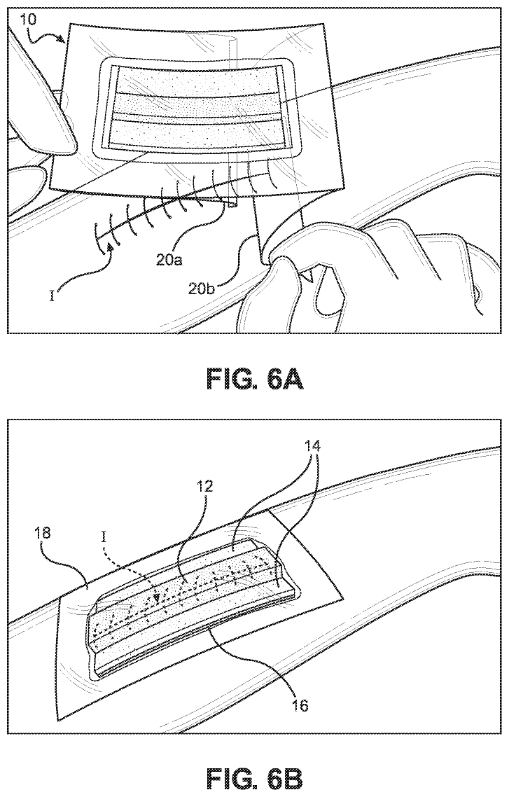

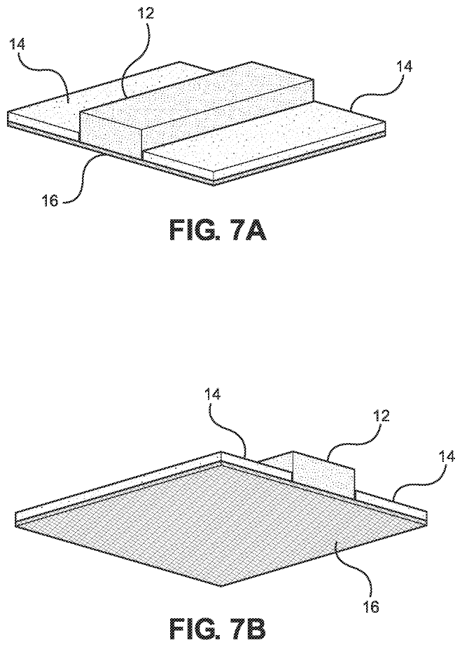

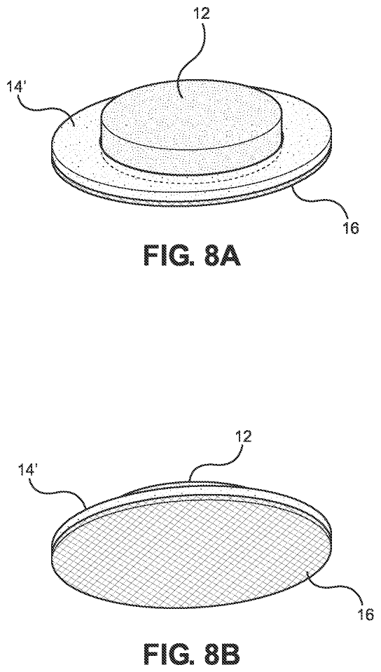

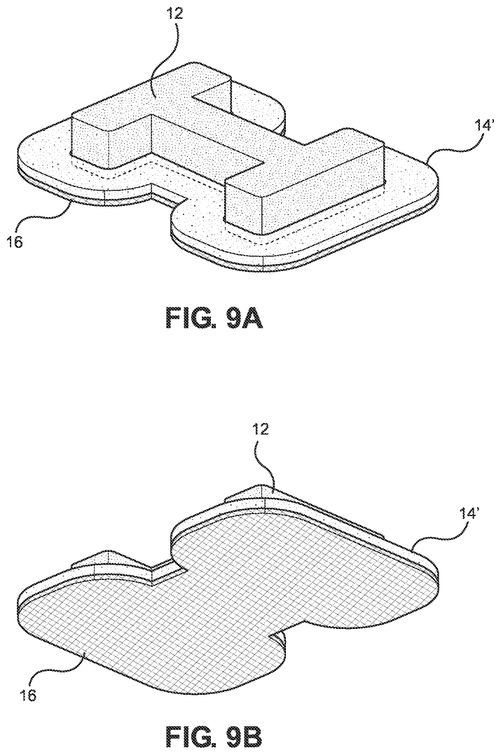

The wound dressing 10 as depicted and described in FIGS. 1-6B is configured as a rectangle. However, it will be understood that the dressing 10 can be configured in a plethora of other shapes including but not limited to square and circular, such as shown in FIGS. 7A-8B. Additional shape examples are also shown in FIGS. 9A-15B.

The primary components of the incision dressing 10 as shown are an elongate liquid permeable center section 12 and lateral absorbent sections 14 that abut the center section 12 on each elongate side of the center section 12. To keep the center section 12 and the lateral absorbent sections 14 in place relative to one another and to provide a soft skin contact surface, a wound contact material 16 is configured in the footprint of the center section 12 and the lateral absorbent sections 14 and adhered to the lowermost surfaces thereof.

The liquid permeable center section 12 is placed to overlie the incision I or other wound. The wound contact material 16 underlies the liquid permeable center section 12/As configured, the center section 12 is elongate and narrow to be just slightly greater in length and width than the length and width of the incision I. The liquid permeable center section 12 is preferably made of a hydrophobic, porous, and fluid permeable foam material having a thickness of at least about 0.25 inches. As shown, a black foam is utilized.

Most preferably, this fluid permeable foam material of the center section 12 is a reticulated polyurethane foam with a pores per inch range of 40-50. The center section 12 is desirably thicker than the lateral absorbent sections 14 for use of the dressing 10 in circumstances where a negative pressure is applied to the dressing 10 as part of wound treatment therapy. The thickness of the center section 12 for such uses is selected to be sufficiently thick to ensure there is enough cross-sectional area when the foam is under vacuum to be able to transport fluid and negative pressure effectively.

The lateral absorbent sections 14 are thinner than the center section 12, with a thickness of about 0.04 inches. This thinness of the lateral absorbent sections 14 aids in conforming the dressing 10 to the site of the incision I. The lateral absorbent sections 14 are constructed from an absorbent cellulose fiber material configured to absorb and contain wound exudate and like fluids from the incision I. If desired, the lateral absorbent sections 14 may include lyocell fibers, which are cellulose fibers prepared from cellulose solutions in an organic solvent, and having enhanced absorbency as compared to non-lyocell cellulose fibers. The lateral absorbent sections 14 may each be provided as by 7.9 ounce lyocell pads, having an absorbent capacity of about 65 ounces per square yard.

The wound contact material 16 is a non-adherent wound contact material, preferably made of a non-adherent fluid permeable material such as polyester. Polyester is desirable to allow for both horizontal and vertical wicking of fluid from the incision I to the lateral absorbent section 14 on either side of the incision I.

An uppermost surface 16a of the wound contact material 16 is desirably coated with an adhesive of a type commonly used with wound dressings for securement of the material 16 to the central section 12 and the lateral absorbent sections 14.

The wound contact material 16 provides a non-adherent wound contact material that serves to maintain the relative positions and relationships of the central section 12 and the lateral absorbent sections 14 and does not negatively affect or alter their cooperation with one another.

The center section 12 advantageously provides a flow path of fluid from the incision I and the lateral absorbent sections 14 absorb fluid from the center section 12 and draw fluid to flow from the incision 12 and flow to and through the center section 12 to the lateral absorbent sections 14 for collection of fluids from the incision I. The wound contact material 16 as installed on the center section 12 and the lateral absorbent sections 14 provides a non-adherent wound contact material that serves to maintain the relative positions and relationships of the central section 12 and the lateral absorbent sections 14 and does not negatively affect or alter their cooperation with one another.

The composite provided by the center section 12 and lateral absorbent sections 14, as secured together by the wound contact material 16 is suitable by itself as the incision dressing 10. It may be secured in place as by paper tape or the like. However, in a preferred embodiment, an adhesive polyurethane film 18 is secured over the center section 12 and the lateral absorbent sections 14.

The adhesive polyurethane film 18 is advantageously breathable and transparent and provides at the top of the dressing 10 a barrier against fluid leakage from the dressing 10 and a barrier against bacteria to the incision 10 via the dressing 10. A lowermost surface 18a of the film 18 is desirably coated with an adhesive of a type commonly used with wound dressings.

Release liners 20a and 20b are secured to the bottom of the dressing 10 and secured in place by contact of upper surfaces of the liners 20a and 20b with adhesive of exposed portions of the lowermost surface 18a of the film 18 surrounding the center section 12 and the lateral absorbent sections 14. The release liners 20a and 20b may be conventional release liners used with bandages, typically being made of thin sheet paper with the surface in contact with the adhesive of the lowermost surface 18a being coated with a release coating such as silicone or other release coatings used for medical dressings.

FIGS. 6A-6B show one example of application of the dressing 10 to a wound site, such as the incision I. In this example, the dressing 10 includes the adhesive polyurethane film 18 over the central section 12 and the lateral absorbent sections 14, with the release liners 20a and 20b installed. Initially, the dressing 10 is aligned with the incision I so that the central section 12 is aligned with and overlies the incision I. Next, the rearmost release liner, in this case the liner 20 b, is removed and the exposed film 18 is pressed against the skin of the patient behind the incision I. The release liner 20a is then removed and the remaining exposed film 18 is pressed against the skin of the patient surrounding the incision I. As will be appreciated, the adhesive of the film 18 maintains the dressing 10 in place.

Over time, the dressing 10 will function to cover the incision 10 and enable exudate from the incision I to pass through the central section 12 and be absorbed into and collected by the absorbent lateral sections 14.

FIGS. 7A and 7B depict the dressing 10 configured in a shortened, square configuration.

FIGS. 8A and 8B depict the dressing 10 configured in a circular configuration. The center section 12 is circular. As will be observed, instead of having two lateral absorbent sections 14, there is an absorbent section 14′ that surrounds the circular center section 12. In this embodiment the absorbent section 14′ is a single ring-shaped section. The ring-shaped absorbent section 14′ is configured of the same materials as described for the lateral absorbent sections 14.

FIGS. 9A and 9B depict the dressing 10 in an I-shape, with absorbent sections 14′ surrounding the I-shaped center section 12.

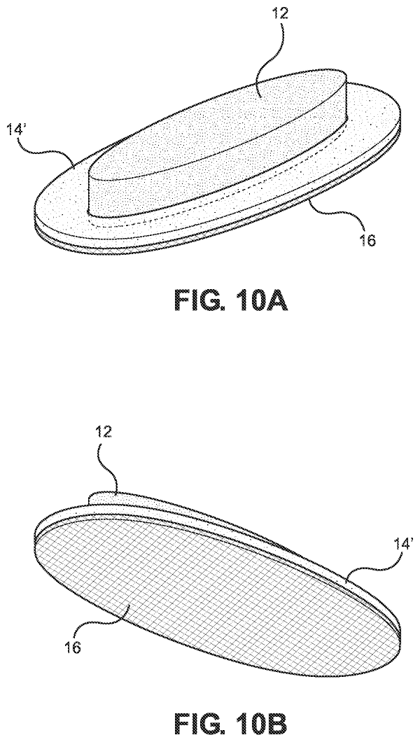

FIGS. 10A and 10B depict the dressing 10 in an oval shape, with an absorbent section 14′ surrounding the oval center section 12.

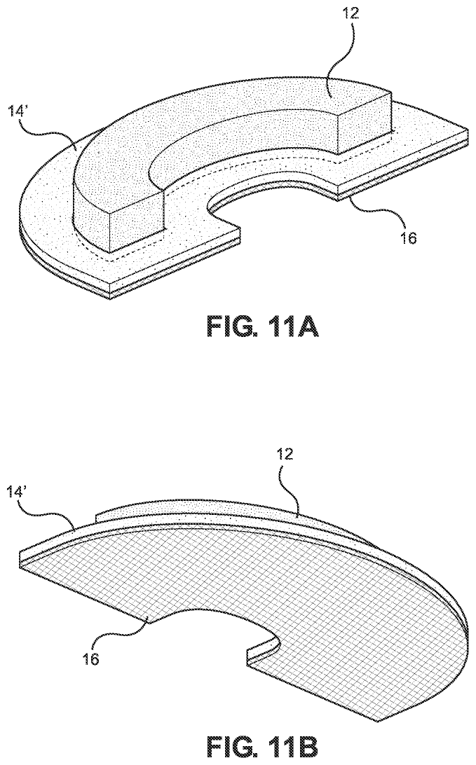

FIGS. 11A and 11B depict the dressing 10 in a C-shape, with absorbent sections 14′ surrounding the center section 12. In embodiments like this and the others described herein, in which the center section 12 is completely surrounded, it will be understood that the center sections 14′ may be provided as a single piece configured to surround the center section 12, or the center sections 14′ may be provided by a plurality of pieces configured to surround the center section 12.

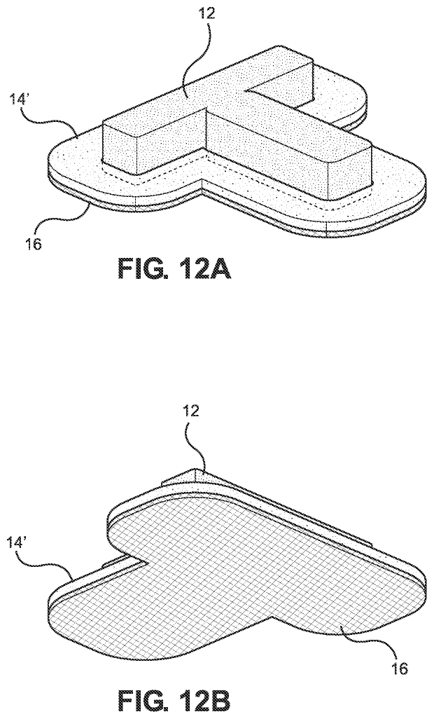

FIGS. 12A and 12B in a T-shape, with absorbent sections 14′ surrounding the center section 12.

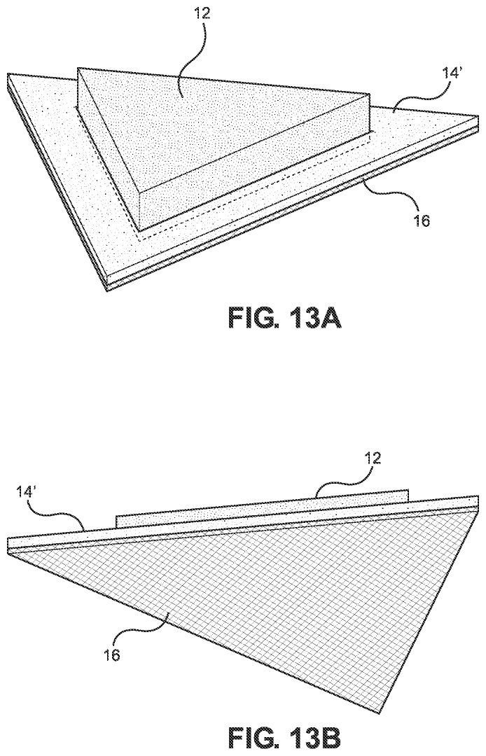

FIGS. 13A and 13B in a triangular shape, with absorbent sections 14′ surrounding the center section 12.

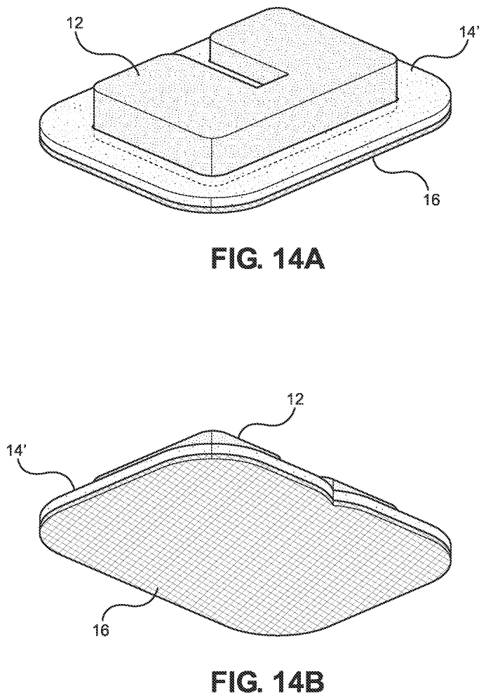

FIGS. 14A and 14B in a U-shape, with absorbent sections 14′ surrounding the center section 12.

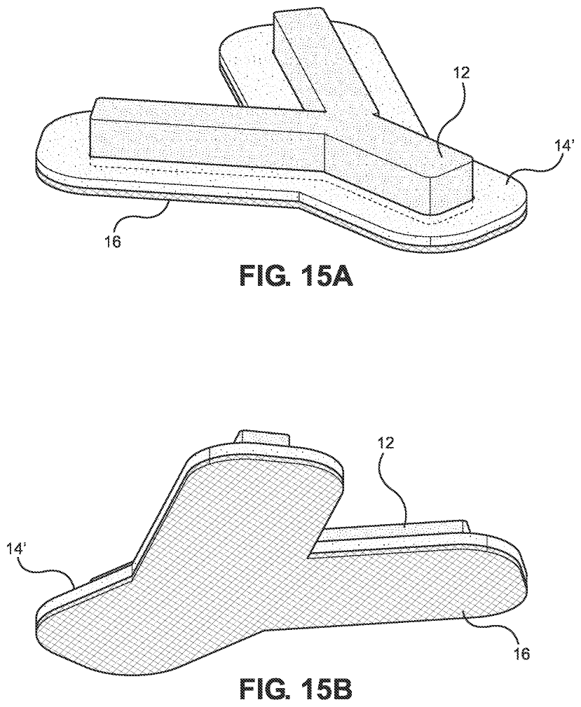

FIGS. 15A and 15B in a Y-shape, with absorbent sections 14′ surrounding the center section 12.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated.

1. An incision dressing, comprising:
a liquid permeable center section;
a pair of lateral absorbent sections abutting the center section on each side of the center section; and
a wound contact material adhered to lowermost surfaces of the center section and the lateral absorbent sections.

2. The incision dressing of claim 1, wherein the liquid permeable center section comprises a hydrophobic, porous, and fluid permeable foam material.

3. The incision dressing of claim 1, wherein the lateral absorbent sections comprise absorbent cellulose fiber materials configured to absorb and contain fluids.

4. The incision dressing of claim 1, wherein the wound contact material comprises a polyethylene fiber mesh.

5. The incision dressing of claim 1, further comprising an adhesive film secured over the center section and the lateral absorbent sections.

6. An incision dressing of claim 5, wherein the adhesive film comprises a polyurethane film.

7. The incision dressing of claim 5, wherein the adhesive film has exposed adhesive surfaces surrounding the center section and the lateral absorbent sections and the dressing further includes release liners secured to a bottom of the dressing by contact of upper surfaces of the release liners with the exposed adhesive surfaces of the adhesive film.

8. The incision dressing of claim 1, wherein the center section has a thickness and the lateral absorbent sections each have a thickness that is less than the thickness of the center section.

9. The incision dressing of claim 1, wherein when the dressing is applied to an incision, the dressing is applied over the incision so that the center section is aligned with and overlies the incision so that fluidic exudate from the incision passes through the center section and is absorbed into and collected by the absorbent lateral sections.

10. The incision dressing of claim 1, wherein the center section and the absorbent lateral sections each have a length and the length of the center section and the absorbent lateral sections is the same.

11. An incision dressing, comprising:
a liquid permeable center section;
an absorbent section abutting and surrounding the center section; and
a wound contact material adhered to lowermost surfaces of the center section and the absorbent section.

12. The incision dressing of claim 11, wherein the center section is circular and the absorbent section is ring-shaped.
